(19) United States
(12) Patent Application Publication
(10) Pub. No.: US 2026/0014020 A1
(43) Pub. Date: Jan. 15, 2026

(54) MULTI-USE INJECTOR

(71) Applicant: Alcon Inc., Fribourg (CH)

(72) Inventors: Adi ARSLANAGIC, Fort Worth, TX (US); Michael PIAZZA, Crowley, TX (US); Marcos ZAMORANO, Fort Worth, TX (US); Reginaldo KUHN, Fort Worth, TX (US)

(21) Appl. No.: 19/257,869

(22) Filed: Jul. 2, 2025

Related U.S. Application Data
(60) Provisional application No. 63/669,217, filed on Jul. 9, 2024.

(51) Int. Cl. A61F 9/007

(52) U.S. Cl. CPC A61F 9/00736

(57) ABSTRACT

The present disclosure provides sterilizable and re-usable devices for injecting substances including, without limitation, viscoelastics, liquids, gels, solutions, emulsions, suspensions, other fluids, and the like, during surgical procedures.

### CROSS-REFERENCE TO RELATED APPLICATIONS

This application claims priority to and benefit of U.S. Provisional Application No. 63/669,217, filed Jul. 9, 2024, which is hereby assigned to the assignee hereof and hereby expressly incorporated by reference in its entirety as if fully set forth below and for all applicable purposes.

### BACKGROUND

Many surgical procedures involve the injection of substances into a body structures. For example, during various ophthalmic surgical procedures, an ophthalmic viscoelastic device (OVD), or viscoelastic substance, may be injected into a patient's eye. In one particular example, an OVD may be injected into the anterior chamber of the patient's eye during cataract surgery to protect intraocular tissues against surgical trauma, absorb ultrasound energy, coat the intraocular lens, and maintain the volume and shape of the anterior chamber. However, OVDs are also commonly used during glaucoma, cornea, and vitreoretinal surgical procedures.

OVDs, as well as other treatment substances, are often introduced into target sites via disposable, single-use syringes and cannulas. These single-use syringes and cannulas are typically individually packaged to maintain sterility and to satisfy procedural conditions. And, in many cases, two or more syringes and cannulas are employed in order to inject a desired amount of treatment substance(s) into a target site, and/or to reapply the treatment substance(s) throughout the surgical procedure. As a result, these products and their corresponding packaging contribute a significant amount of medical waste to each procedure. Even further, such products utilize a significant amount of sterilization resources during fabrication and packaging, and take up significant amounts of space during transportation and storage. Still further, as a plurality of single-use syringes and cannulas may be needed during a single surgical procedure, the set-up of multiple syringes and cannulas may cause procedural inefficiencies.

### SUMMARY

Embodiments of the present disclosure provide a sterilizable and re-usable apparatus for injecting substances (including, without limitation, viscoelastics, liquids, gels, solutions, emulsions, suspensions, other fluids, and the like) during surgical procedures.

In certain embodiments, a re-usable injection device is provided, the injection device comprising: a body, comprising: a compartment disposed within the body and configured to receive a cartridge containing one or more substances; a connector disposed at a distal end of the body and configured to simultaneously receive a hub of a cannula and a nozzle of the cartridge to facilitate connection of the cannula and the cartridge when the cartridge is disposed in the compartment, the connector further comprising a feature for mating with the hub to secure the cannula to the connector when the cannula is connected to the cartridge; and a finger flange disposed at a proximal end of the body, the finger flange configured to contact a flange of the cartridge when the cartridge is disposed in the compartment; and a plunger assembly coupled to the proximal end of the body, the plunger assembly comprising: a cap configured to secure the cartridge when the cartridge is disposed in the compartment; and a plunger movably disposed through the cap and configured to engage with a piston of the cartridge to dispense fluids from the cartridge when actuated by a user.

In certain embodiments, a re-usable injection device is provided, the injection device comprising: a body, comprising: a compartment disposed within the body and configured to receive a cartridge containing one or more substances; a connector disposed at a distal end of the body and configured to simultaneously receive a hub of a cannula and a nozzle of the cartridge to facilitate connection of the cannula and the cartridge when the cartridge is disposed in the compartment; a finger flange disposed at a proximal end of the body, the finger flange comprising a slot configured to receive and secure a flange of the cartridge when the cartridge is disposed in the compartment; an opening disposed at the proximal end of the body and configured to accommodate a plunger of the cartridge; and a window extending along a length of the body, the window configured to facilitate insertion of the cartridge into the compartment and visual inspection of the cartridge when the cartridge is disposed in the compartment.

In certain embodiments, a re-usable injection device is provided, the injection device comprising: a body, comprising: a compartment disposed within the body and configured to receive a cartridge containing one or more substances; and a finger flange disposed at a proximal end of the body, the finger flange configured to contact a flange of the cartridge when the cartridge is disposed in the compartment; a plunger assembly coupled to the proximal end of the body, the plunger assembly comprising: a cap fixedly coupled to the body; and a plunger movably disposed through the cap and configured to engage with a piston of the cartridge to dispense fluids from the cartridge when actuated by a user; and a connector configured to removably couple to a distal end of the body, the cap configured to facilitate connection of a cannula with the cartridge when the cartridge is disposed in the compartment, the connector further comprising a feature for mating with the cannula to secure the cannula to the connector when the cannula is connected to the cartridge.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is a perspective side view of a re-usable apparatus for injecting substances, according to certain embodiments of the present disclosure.

FIG. 1B is another perspective side view of the apparatus of FIG. 1A, according to certain embodiments of the present disclosure.

FIG. 2A is a perspective side view of a re-usable apparatus for injecting substances, according to certain embodiments of the present disclosure.

FIG. 2B is another perspective side view of the apparatus of FIG. 2A, according to certain embodiments of the present disclosure.

FIG. 3A is a perspective side view of a re-usable apparatus for injecting substances, according to certain embodiments of the present disclosure.

FIG. 3B is another perspective side view of the apparatus of FIG. 3A, according to certain embodiments of the present disclosure.

FIG. 4A is a perspective side view of a re-usable apparatus for injecting substances, according to certain embodiments of the present disclosure.

FIG. 4B is another perspective side view of the apparatus of FIG. 4A, according to certain embodiments of the present disclosure.

FIG. 5A is a perspective side view of a re-usable apparatus for injecting substances, according to certain embodiments of the present disclosure.

FIG. 5B is another perspective side view of the apparatus of FIG. 4A, according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

As described above, OVDs and other treatment substances are often introduced into body structures, such the intraocular space of the eye, via single-use syringes and cannulas. These single-use syringes and cannulas are typically individually packaged to maintain sterility and to satisfy procedural conditions. And, in many cases, two or more syringes and cannulas are employed in a single surgical procedure.

As a result, these products and their corresponding packaging contribute a significant amount of medical waste to each procedure. Such medical waste is subject to strict regulations, and must typically be treated with chemicals, autoclaving, incineration, or other approved methods before disposal. Excess medical waste can have many negative environmental implications, including the release of toxic pollutants and pathogens. For example, if surgical waste is treated with chemical disinfectants, these substances can be inadvertently released into the environment if the waste is not properly disposed of thereafter. And while incineration is widely used to dispose of surgical waste, this practice can result in the release of pollutants in the air if it is not done properly, or if improper materials are incinerated.

With single-use syringes, cannulas, and related devices, healthcare providers must also rely on receiving a constant, uninterrupted supply of the devices they need to ensure that they have enough stock for their routine operations. In some cases, the constant demand for single-use devices can put a healthcare provider at risk of having to delay or cancel procedures if there are any product supply issues. To avoid disruptions to their routine operations, healthcare providers often overstock their supply of single-use devices, which can occupy a significant amount of storage space.

Further, the constant demand for single-use syringes and cannulas causes a corresponding drain on sterilization resources, as each device needs to be sterilized during fabrication and/or packaging. For example, one commonly utilized sterilizing agent for medical devices is ethylene oxide (EtOH) gas, which is a known carcinogen.

There is therefore a need for sterilizable and re-usable devices for injecting substances (including, without limitation, viscoelastics, liquids, gels, solutions, emulsions, suspensions, other fluids, and the like) during surgical procedures. Embodiments described herein provide examples of such devices.

In certain embodiments, the devices disclosed herein can be utilized for injection or application of ophthalmic substances into and/or onto a patient's eye. Examples of ophthalmic substances include, without limitation, ophthalmic viscoelastic devices (OVDs), ophthalmic medicaments such as vascular endothelial growth factor (VEGF) inhibitors, antibiotics, anesthetics, and steroids, other ophthalmic gels and solutions, and the like. However, the utilization of such devices with other types of treatment substances and/or for injection or application of other body structures is also contemplated.

Examples will now be described relative to the Drawings.

In the description herein, “proximal” with reference to an injector or components thereof shall be understood as including, or being nearest, an end opposite of a dispensing end of the injector. “Distal” with reference to the injector or components thereof shall be understood as including, or being nearest, an end from which substances are dispensed from the injector.

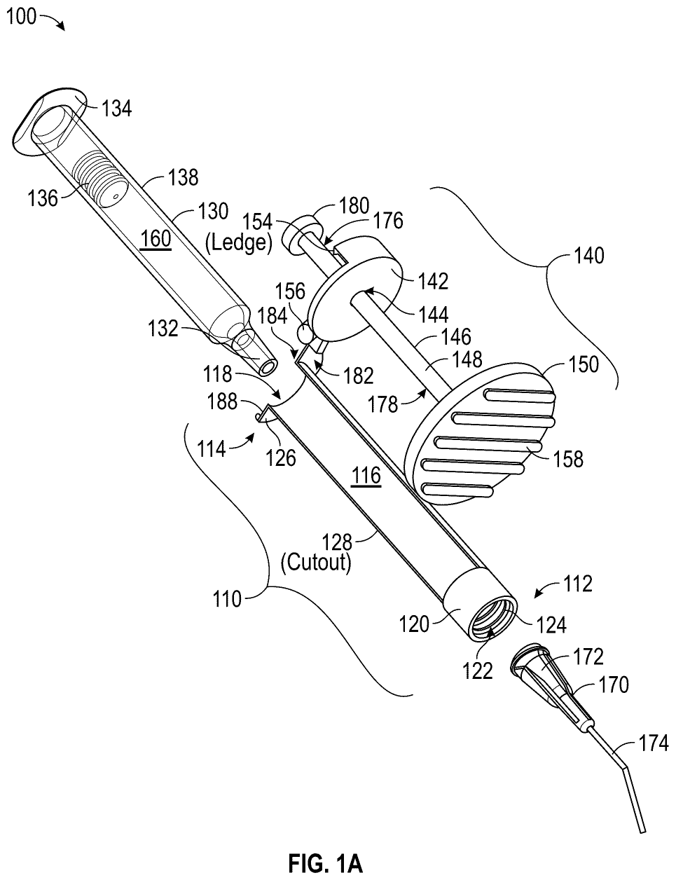

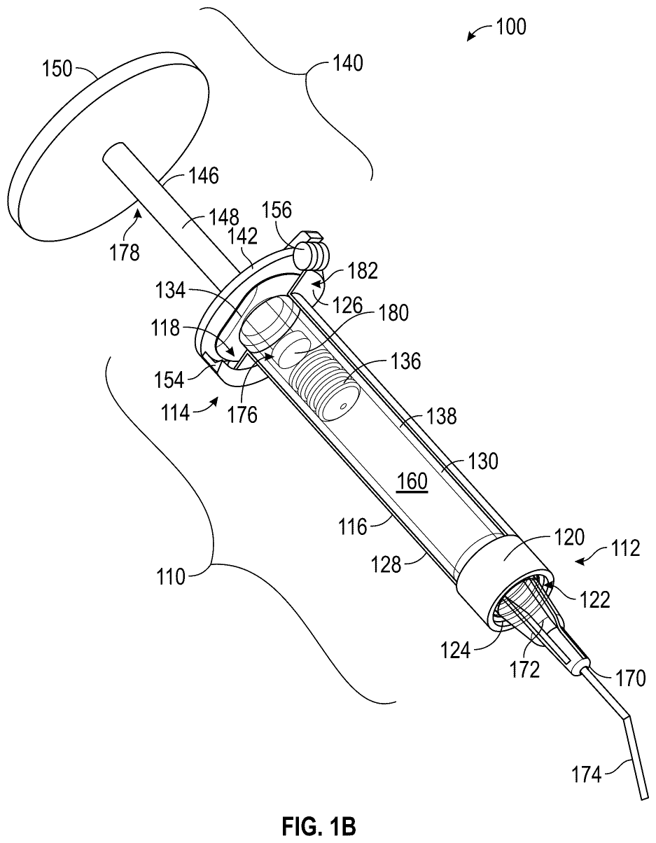

FIG. 1A is a perspective side view of an injector device 100 for injecting substances, according to certain embodiments of the present disclosure. FIG. 1B is another perspective side view of the injector device 100, according to certain embodiments of the present disclosure. In FIG. 1A, the injector device 100 is shown in an unassembled, or disassembled, state; meanwhile, in FIG. 1B, the injector device 100 is shown in an assembled state. In both FIGS. 1A and 1B, the injector device 100 is shown relative to a cartridge 130 containing a substance for injection and a corresponding cannula 170. For clarity, FIGS. 1A and 1B are described together herein.

As shown in FIGS. 1A and 1B, the injector device 100 includes a body 110 and a plunger assembly 140. The body 110 is configured to receive the cartridge 130 therein, while the plunger assembly 140 is configured to secure the cartridge 130 in the body 110 and facilitate dispensing of the substance from the cartridge 130.

In the illustrated embodiments, the body 110 has a generally tubular or cylindrical shape extending between a distal end 112 and a proximal end 114 of the body 110. It should be appreciated that the shape shown relative to the body 110 is merely illustrative. In various embodiments, the body 110 can have any suitable geometry, shape, and/or size including, for example, tapered shapes, ovaloid shapes, and/or the like. The body 110 is formed of any suitable material that facilitates sterilization and re-use of the injector device 100. In certain embodiments, the body 110 is formed of a surgical grade metal or metal alloy, such as stainless steel, titanium, tungsten carbide, carbon steel, and the like. In certain embodiments, the body 110 is formed of a sterilizable thermoplastic or resin-based material.

The body 110 includes a compartment 116 formed therein for receiving the cartridge 130. In certain embodiments, the compartment 116 extends from a proximal opening 118 at the proximal end 114 of the body 110 to a distal opening 122 at the distal end 112, or a point near the distal end 112. The compartment 116 is generally shaped and sized to correspond with a shape and size of the cartridge 130. Accordingly, the compartment 116 may have inner dimensions (e.g., an inner diameter, width, or length) substantially similar to outer dimensions (e.g., an outer diameter, width, or length) of the cartridge 130 to facilitate a secure fit of the cartridge 130 within the body 110. In the embodiments of FIGS. 1A and 1B, the cartridge 130 can be received by the compartment 116 through the proximal opening 118. Accordingly, the proximal opening 118 may have a cross-section (e.g., cross-sectional size and shape) similar to that of the compartment 116 and/or the cartridge 130.

The body 110 also includes a connector 120 disposed at the distal end 112 of the body 110 that forms the distal opening 122 extending between the compartment 116 and an exterior of the body 110. In certain embodiments, the connector 120 is simply an extension of the body 110. The connector 120 facilitates fluid connection of the cartridge 130 to the cannula 170 when the cartridge 130 is fully received in the compartment 116. Accordingly, the connector 120 is configured to receive at least a portion of a nozzle 132 of the cartridge 130 and at least a portion of a hub 172 of the cannula 170 through the distal opening 122. In certain embodiments, the distal opening 122 has inner dimensions (e.g., an inner diameter or width) that are smaller than those of the compartment 116 to facilitate a secure fit of the nozzle 132 and hub 172. In certain embodiments, the connector 120 includes one or more threads 124 and/or other features disposed along inner surfaces of the connector 120 and surrounding the distal opening 122. In such embodiments, the thread(s) 124 and/or other feature(s) of the connector 120 are configured to mate with corresponding features on the hub 172 to secure the cannula 170 to the body 110 while the cannula 170 is connected to the cartridge 130 received in the compartment 116. In certain embodiments, fully securing the cannula 170 against the connector 120 may produce an audible indication for the user that confirms proper fastening of the components.

In certain embodiments, in addition to the connector 120, the injector device 100 may include a secondary locking mechanism for the cannula 170. For example, the injector device 100 may include a claw or clamp that is disposed around the distal end of the connector 120 and that when compressed, enables the cannula 170 to be inserted into the distal opening 122. When the claw or clamp is released, the claw or clamp “hugs” the cannula 170 to secure the cannula 170 in the distal opening 122 and prevent the cannula 170 from distally separating from the injector device 100.

The body 110 further includes one or more body flanges 126 disposed at or near the proximal end 114 and extending laterally, or radially, outward from the body 110. A distal surface 182 of each body flange 126 facilitates grasping of the injector device 100 by the fingers of a surgeon or surgical assistant during a surgical operation, such as a cataract procedure or other ophthalmic procedure. Thus, in certain examples herein, a body flange 126 may be referred to as a “flinger flange.” A proximal surface 184 of each body flange 126 may further be configured to contact and support a flange 134 of the cartridge 130 when the cartridge 130 is fully received in the compartment 116, and to prevent further distal movement of the cartridge 130 through the body 110 during assembly. In certain embodiments, a singular body flange 126 is disposed around an entire cross-sectional outer perimeter (e.g., circumference) of the body 110; in certain other embodiments, one or more body flanges 126 are each disposed around only a portion of the cross-sectional outer perimeter (and/or proximal opening 118) of the body 110.

In certain embodiments, a window 128 extends along a length of the body 110. The window 128 facilitates viewing by the surgeon or surgical assistant of the cartridge 130 disposed in the injector device 100, a type of substance disposed in the cartridge 130, and/or an amount of substance remaining in the cartridge 130, when the cartridge 130 is received in the compartment 116. In certain embodiments, the window 128 further facilitates insertion of the cartridge 130 into the injector device 100. In the embodiment of FIGS. 1A and 1B, the window 128 extends from the proximal end 114 of the body 110 to the connector 120, and aligns with or forms a cutout 188 in the singular body flange 126. However, other arrangements of the window 128 are also contemplated. Further, it is also contemplated that the body 110 may be formed of a transparent or translucent material in certain embodiments to facilitate visibility of an inserted cartridge 130 with or without the use of a window.

In certain embodiments, a shape of the body 110 (or another component of the injector device 100) indicates or represents the type of cartridge 130 for use with the injector device 100, and/or a type of substance in the cartridge 130. In certain embodiments, the body 110 (or another component of the injector device 100) includes one or more visual indicators that indicate, or represent, the type of cartridge 130 and/or substance in the cartridge 130. For example, the body 110 can include a marking or other graphic on an external surface thereof to indicate the type of cartridge 130 and/or substance disposed therein. In certain embodiments, one or more components of the injector device 100 are color-coded to indicate the type of cartridge 130 and/or substance disposed therein. For example, the body 110 can include a color-coded ring (e.g., disposed around an external surface thereof) to indicate the type of cartridge 130 and/or substance for use with the injector device 100.

The plunger assembly 140 includes an endcap 142 and a plunger 146 movably disposed through the endcap 142. Similar to the body 110, the endcap 142 and the plunger 146 are formed of any suitable material(s) that facilitate sterilization and re-use of the injector device 100. In certain embodiments, the endcap 142 and/or the plunger 146 are formed of a surgical grade metal or metal alloy, such as stainless steel, titanium, tungsten carbide, carbon steel, and the like. In certain embodiments, the endcap 142 and/or the plunger 146 are formed of a sterilizable thermoplastic or resin-based material. In certain embodiments, the body 110, the endcap 142, and the plunger 146 are formed of the same material. In certain other embodiments, the body 110, the endcap 142, and/or the plunger 146 are formed of a different material from one or both of the other components.

The endcap 142 includes a suitably-shaped capping member configured to fasten against the body flange 126 upon insertion of the cartridge 130 into the compartment 116. In the embodiments of FIGS. 1A and 1B, the endcap 142 includes a disk-like capping member having an aperture 144 through which the plunger 146 is slidably disposed. Fastening the endcap 142 against the body flange 126 secures the cartridge 130 within the body 110, and further positions the plunger 146 to engage with a piston 136 of the cartridge 130. In certain embodiments, the endcap 142 includes one or more locking features 154 for securing the endcap 142 to the body flange 126. Such locking features 154 may include snap-fit protrusions, grooves, and the like, which may be formed on an internal or external surface of the endcap 142 and configured to engage with corresponding features on the body flange 126, such as an outer edge of the body flange 126. In certain embodiments, fully securing the endcap 142 against the body flange 126 may produce an audible indication for the user that confirms proper fastening of the components.

In certain embodiments, the endcap 142 is movably secured to the body 110 to prevent unintentional separation and misplacement of the plunger assembly 140 and/or body 110. For example, in the embodiments of FIGS. 1A and 1B, the endcap 142 is hingedly coupled with the body flange 126 via a hinge 156. Accordingly, the plunger assembly 140 and/or body 110 may be rotated about an axis of the hinge 156 to fasten and unfasten the plunger assembly 140 and the body 110 together during assembly or disassembly. However, other types of movable joints and mechanisms are also contemplated for securing the plunger assembly 140 to the body 110.

As shown in FIGS. 1A and 1B, the plunger 146 includes a rod 148 and a plunger flange 150 fixedly coupled to a proximal end 178 of the rod 148. The rod 148 includes an elongated member configured to slide axially through the aperture 144 of the endcap 142 upon actuation by a user. When the endcap 142 is fastened against the body flange 126, a distal end 176 of the rod 148 is disposed within the compartment 116 of the body 110 to facilitate engagement of the rod 148 with the piston 136 of the cartridge 130 for dispensing fluids from the cartridge 130, as described in more detail below. In certain embodiments, the distal end 176 of the rod 148 includes a stopper 180, or flange, for preventing the plunger 146 from sliding proximally out of the aperture 144, as shown in FIG. 1B. In such embodiments, the stopper 180 may directly engage with the piston 134 of the cartridge 130 during use.

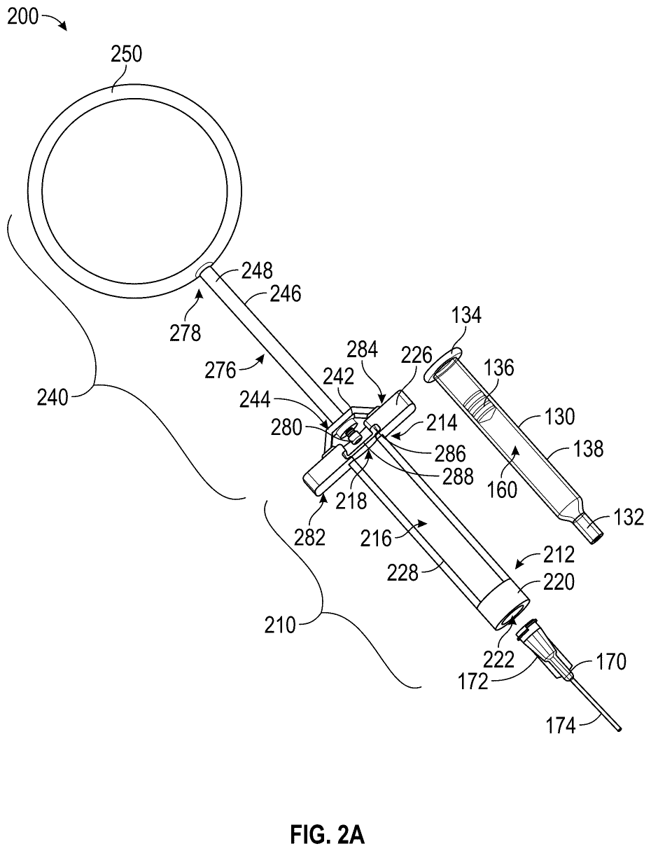

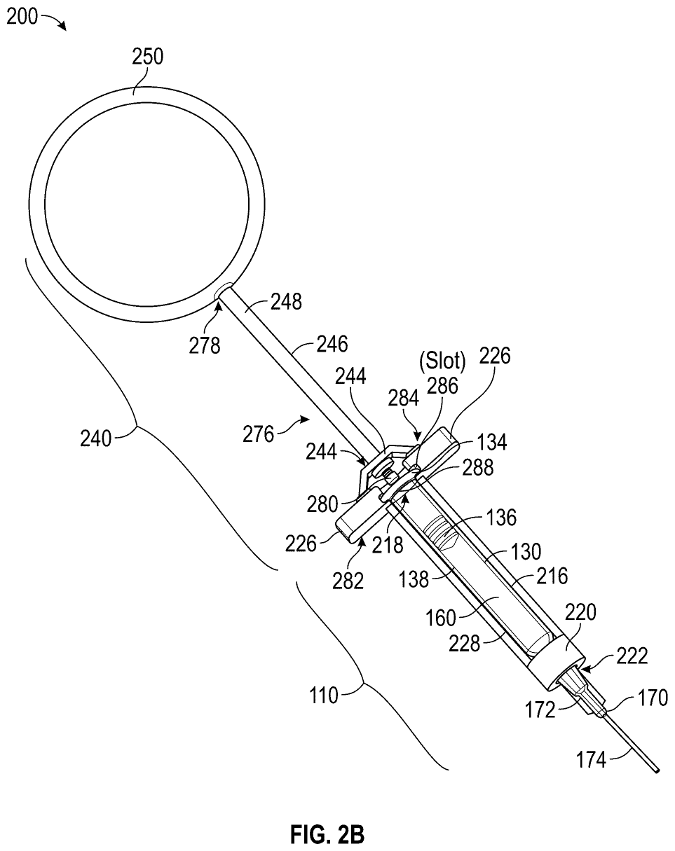

The plunger flange 150 provides a surface upon which a user (e.g., a surgeon) may press to actuate the rod 148 through the body 110. Typically, the user will press against the plunger flange 150 with their thumb, and so the plunger flange 150 may be referred to as a “thumb flange” in certain examples herein. In certain examples, the plunger flange 150 may include one or more textured, contoured, and/or curved surfaces 158 for improved gripping thereof by the user. In still further embodiments, the plunger flange 150 may be replaced by another feature to facilitate actuation of the rod 148, such as a ring as shown in the embodiments of FIGS. 2A and 2B.

The cartridge 130 can include any suitable type of cartridge or receptacle for storing and delivering substances for injection. In certain embodiments, such as shown in FIGS. 1A and 1B, the cartridge 130 resembles a syringe. However, other types of cartridges 130 are also contemplated. Generally, the cartridge 130 includes a body 138, the nozzle 132 at a distal end of the body 138, the flange 134 at a proximal end of the body 138, and the piston 136 disposed within an internal compartment 160 of the body 138. As noted above, the nozzle 132 connects, or mates, with the hub 172 of a cannula 170 through the connector 120 of the body 110. The flange 134 contacts against the proximal surface 184 of the body flange 126 to help secure the cartridge 130 in place within the compartment 116. Further, the piston 136 engages with the rod 148 and/or the stopper 180 of the plunger 146 to facilitate dispensing of substances from the cartridge 130, and thus, the injector device 100.

In certain embodiments, the type or shape of the cartridge 130 indicates or represents the type of substance contained within the cartridge 130. In certain embodiments, the cartridge 130 includes one or more visual indicators that indicate, or represent, the type of substance contained within the cartridge 130. For example, the cartridge 130 can include a marking or other graphic on an external surface thereof to indicate the type of substance contained therein. In certain embodiments, one or more components of the cartridge 130 are color-coded to indicate the type of substance contained within the cartridge 130. For example, the cartridge 130 can include a color-coded ring around an external surface thereof to indicate the type of substance contained therein. Generally, visual indicators and/or color-coded components on the cartridge 130 may be visible through the window 128 of the body 110 to facilitate viewing by the surgeon or surgical assistant when the cartridge 130 is disposed in the injector device 100.

The cannula 170 includes the hub 172 and a needle 174 through which substances from the cartridge 130 are flowed during injection. Generally, the cannula 170 may include any suitable cannula type and size, such as a cannula having a 20-gauge, 21-gauge, 22-gauge, 23-gauge, 24-gauge, 25-gauge, 26-gauge, 27-gauge, 28-gauge, 29-gauge, 30-gauge, 31-gauge, 32-gauge, 33-gauge, or smaller or larger diameter needle 174.

To assemble and use the injector device 100, a user (e.g., a surgeon or surgical assistant) first inserts the cartridge 130, nozzle 132 first, through the proximal opening 118 and/or window 128 of the body 110 and into the compartment 116. The user then slides the cartridge 130 distally through the compartment 116 such that the nozzle 132 is received in the connector 120. Engagement of one or more surfaces of the cartridge 130 with the connector 120 or another component of the body 110 may produce an audible indication (e.g., a sound) for the user to confirm proper and complete insertion of the cartridge 130 into the compartment 116. Once the cartridge 130 is fully inserted into the compartment 116 of the body 110, the user flips, or rotates, the plunger assembly 140 about the hinge 156, and secures the endcap 142 of the plunger assembly 140 to the one or more body flanges 126 of the body 110. Again, locking of the endcap 142 against the body flange(s) 126 may produce an audible indication for the user to confirm proper fastening of the components.

Prior to or after insertion of the cartridge 130 and fastening of the plunger assembly 140 and the body 110, the user secures the cannula 170 to the connector 120 of the body 110. For example, the user may screw the cannula 170 into the connector 120 in examples where the connector includes threads 124. Similar to the above, engagement of one or more surfaces of the cannula 170 with the connector 120 may produce an audible indication for the user to confirm proper fastening of the cannula 170 into the connector 120, and/or proper attachment of the cannula 170 with the cartridge 130.

Once the cartridge 130 is inserted within the compartment 116, the plunger assembly 140 is fastened against the body 110, and the cannula 170 is secured within the connector 120, the injector device 100 is ready for use to inject substances from within the cartridge 130 into a body structure of a patient, such as the anterior chamber of a patient's eye. To inject the substances, a user may insert the needle 174 of the cannula 170 into the body structure and adjacent a target injection site, and then press distally against the plunger flange 150 to drive substances from within the cartridge 130 out of the nozzle 132, through the cannula 170, and out of the distal end of the needle 172.

FIG. 2A is a perspective side view of an injector device 200 for injecting substances, according to certain embodiments of the present disclosure. FIG. 2B is another perspective side view of the injector device 200, according to certain embodiments of the present disclosure. In FIG. 2A, the injector device 200 is shown in an unassembled, or disassembled, state; meanwhile, in FIG. 2B, the injector device 200 is shown in an assembled state. In both FIGS. 2A and 2B, the injector device 200 is shown relative to the cartridge 130 containing a substance for injection. For clarity, FIGS. 2A and 2B are described together herein.

Like the injector device 100, the injector device 200 includes a body 210 and a plunger assembly 240. The body 210 is configured to receive the cartridge 130 containing a substance for injection, while the plunger assembly 240 is configured to secure the cartridge 130 in the body 210 and facilitate dispensing of the substance from the cartridge 130.

In the illustrated embodiments, the body 210 has a generally tubular or cylindrical shape extending between a distal end 212 and a proximal end 214 of the body 210. It should be appreciated that the shape shown relative to the body 210 is merely illustrative. In various embodiments, the body 210 can have any suitable geometry, shape, and/or size including, for example, tapered shapes, ovaloid shapes, and/or the like. The body 210 is formed of any suitable material that facilitates sterilization and re-use of the injector device 200. In certain embodiments, the body 210 is formed of a surgical grade metal or metal alloy, such as stainless steel, titanium, tungsten carbide, carbon steel, and the like. In certain embodiments, the body 210 is formed of a sterilizable thermoplastic or resin-based material.

The body 210 includes a compartment 216 for receiving the cartridge 130. In certain embodiments, the compartment 216 extends from a proximal opening 218 at the proximal end 214 of the body 210 to a distal opening 222 at the distal end 212, or a point near the distal end 212. The compartment 216 is generally shaped and sized to correspond with a shape and size of the cartridge 130. Accordingly, the compartment 216 may have inner dimensions (e.g., an inner diameter, width, or length) substantially similar to outer dimensions (e.g., an outer diameter, width, or length) of the cartridge 130 to facilitate a secure fit of the cartridge 130 within the body 210. In the embodiments of FIGS. 2A and 1B, the cartridge 130 can be received by the compartment 216 through a window 228, as further described below.

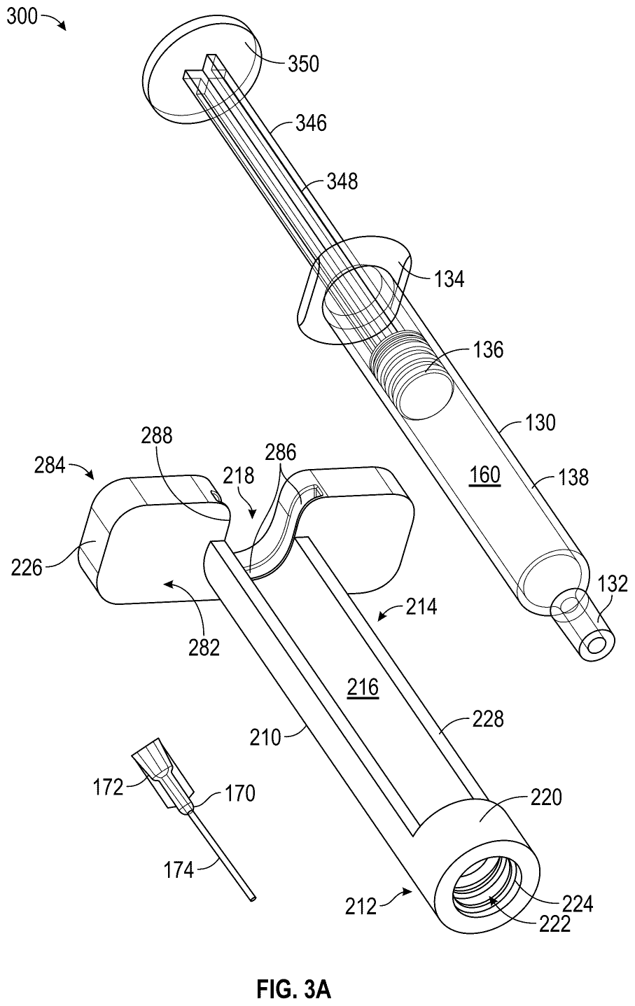

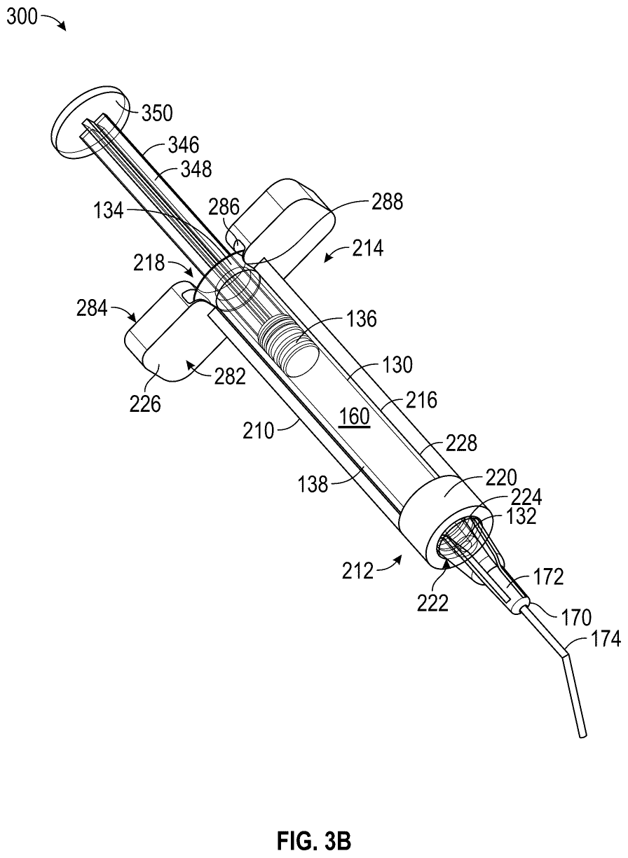

The body 210 also includes a connector 220 disposed at the distal end 212 of the body 210 that forms the distal opening 222 extending between the compartment 216 and an exterior of the body 210. In certain embodiments, the connector 220 is simply an extension of the body 210. The connector 220 facilitates fluid connection of the cartridge 130 to the cannula 170 when the cartridge 130 is fully received in the compartment 216. Accordingly, the connector 220 is configured to receive at least a portion of the nozzle 132 of the cartridge 130 and at least a portion of the hub 172 of the cannula 170 through the distal opening 222. In certain embodiments, the distal opening 222 has inner dimensions (e.g., an inner diameter or width) that are smaller than those of the compartment 216 to facilitate a secure fit of the nozzle 132 and the hub 172. In certain embodiments, the connector 220 includes one or more threads 224 (see FIGS. 3A and 3B for reference) and/or other features disposed along inner surfaces of the connector 220 and surrounding the distal opening 222. In such embodiments, the thread(s) 224 and/or other feature(s) of the connector 220 are configured to mate with corresponding features on the hub 172 to secure the cannula 170 to the body 210 while the cannula 170 is connected to the cartridge 130 received in the compartment 216. In certain embodiments, fully securing the cannula 170 against the connector 220 may produce an audible indication for the user that confirms proper fastening of the components.

In certain embodiments, in addition to the connector 220, the injector device 200 may include a secondary locking mechanism for the cannula 170. For example, the injector device 200 may include a claw or clamp that is disposed around the distal end of the connector 220 and that when compressed, enables the cannula 170 to be inserted into the distal opening 222. When the claw or clamp is released, the claw or clamp “hugs” the cannula 170 to secure the cannula 170 in the distal opening 222 and prevent the cannula 170 from distally separating from the injector device 200.

The body 210 further includes one or more body flanges 226 disposed at or near the proximal end 214 and extending laterally, or radially, outward from the body 210. A distal surface 282 of each body flange 226 facilitates grasping of the injector device 200 by the fingers of a surgeon or surgical assistant during a surgical operation, such as a cataract procedure or other ophthalmic procedure. Thus, similar to the body flange 126, each body flange 226 may be referred to as a “flinger flange” in certain examples herein. However, unlike the body flange 126, the body flange(s) 226 have a slot 286 formed therein for receiving and securing the flange 134 of the cartridge 130 when the cartridge 130 is fully received in the compartment 116. Accordingly, the body flange(s) 226 may have a thickness greater than that of the corresponding body flange 126 of the injector device 100 in order to facilitate the arrangement of the slot 286 therein. Generally, the slot 286 maybe have a size and shape corresponding to a size and shape of the flange 134. In FIGS. 2A and 2B, two body flanges 226 are each disposed around a only portion of the cross-sectional outer perimeter of the body 210 (and/or the proximal opening 218, which extends through or between the body flange(s) 226); however, it is further contemplated that a singular body flange 226 may be disposed around a only portion of the cross-sectional outer perimeter.

In certain embodiments, the window 228 extends along a length of the body 210 to facilitate insertion of the cartridge 130 into the injector device 200. The window 228 further facilitates viewing by the surgeon or surgical assistant of the cartridge 130 disposed in the injector device 100, the type of substance disposed in the cartridge 130, and/or an amount of substance remaining in the cartridge 130, when the cartridge 130 is received in the compartment 216. In the embodiment of FIGS. 2A and 2B, the window 228 extends from the proximal end 214 of the body 210 to the connector 220, and aligns with or forms a cutout 288 (an alternate angle of a corresponding cutout 388 is shown in FIGS. 3A and 3B for reference) in the body flanges 226 that transverses the proximal opening 218. However, other arrangements of the window 228 are also contemplated.

In certain embodiments, a shape of the body 210 (or another component of the injector device 200) indicates or represents the type of cartridge 130 for use with the injector device 200, and/or a type of substance in the cartridge 130. In certain embodiments, the body 210 (or another component of the injector device 200) includes one or more visual indicators that indicate, or represent, the type of cartridge 130 and/or substance in the cartridge 130. For example, the body 210 can include a marking or other graphic on an external surface thereof to indicate the type of cartridge 130 and/or substance disposed therein. In certain embodiments, one or more components of the injector device 200 are color-coded to indicate the type of cartridge 130 and/or substance disposed therein. For example, the body 210 can include a color-coded ring (e.g., disposed around an external surface thereof) to indicate the type of cartridge 130 and/or substance for use with the injector device 200.

The plunger assembly 240 includes a bridge 242 and a plunger 246 movably disposed through the bridge 242. Similar to the body 210, the bridge 242 and the plunger 246 are formed of any suitable material(s) that facilitate sterilization and re-use of the injector device 200. In certain embodiments, the bridge 242 and/or the plunger 246 are formed of a surgical grade metal or metal alloy, such as stainless steel, titanium, tungsten carbide, carbon steel, and the like. In certain embodiments, the bridge 242 and/or the plunger 246 are formed of a sterilizable thermoplastic or resin-based material. In certain embodiments, the body 210, the bridge 242, and the plunger 246 are formed of the same material. In certain other embodiments, the body 210, the bridge 242, and/or the plunger 246 are formed of a different material from one or both of the other components.

The bridge 242 is fixedly, or in certain embodiments, removably, coupled to a proximal surface 284 of each body flange 226 and spans across the proximal opening 218. Generally, the bridge 242 may include any suitably shaped structure or component configured to support and align the plunger 246 with the cartridge 130 when the cartridge 130 is received in the compartment 216. As shown, the bridge 242 has an aperture 244 formed therein and through which the plunger 246 is slidably disposed.

The plunger 246 includes a rod 248 and a ring 250 fixedly coupled to a proximal end 278 of the rod 248. The rod 248 includes an elongated member configured to slide axially through the aperture 244 of the bridge 242 and through the proximal opening 218 of the body 210. Upon distal actuation by a user, a distal end 276 of the rod 248 engages the piston 136 of the cartridge 130 to dispense fluids from the cartridge 130, as described in more detail below. In certain embodiments, the distal end 276 of the rod 248 includes a stopper 280, or flange, for preventing the plunger 246 from sliding proximally out of the aperture 244. In such embodiments, the stopper 280 may directly engage with the piston 134 of the cartridge 130 during use.

The plunger ring 250 provides an interface with which a user (e.g., a surgeon) may actuate the rod 248 through the body 210. In the illustrated examples, the user may insert at least a portion of their thumb through the plunger ring 250 and press distally against an inner surface of the plunger ring 250 to actuate the rod 248, and so the plunger ring 250 may be referred to as a “thumb ring” in certain examples herein. In further embodiments, the plunger ring 250 may be replaced by another feature to facilitate actuation of the rod 248, such as a flange as shown in the embodiments of FIGS. 1A and 1B.

To assemble and use the injector device 200, a user (e.g., a surgeon or surgical assistant) first inserts the cartridge 130, nozzle 132 first, at an angle through window 228 of the body 210 and into the compartment 216. The user then slides the cartridge 130 distally through the compartment 216 such that the nozzle 132 is received in the connector 220. Engagement of one or more surfaces of the cartridge 130 with the connector 220 or another component of the body 210 may produce an audible indication (e.g., a sound) for the user to confirm proper and complete insertion of the cartridge 130 into the compartment 216.

Prior to or after insertion of the cartridge 130, the user secures the cannula 170 to the connector 220 of the body 210. For example, the user may screw the cannula 170 into the connector 220 in examples where the connector includes threads 224. Similar to the above, engagement of one or more surfaces of the cannula 170 with the connector 220 may produce an audible indication for the user to confirm proper fastening of the cannula 170 into the connector 220, and/or proper attachment of the cannula 170 with the cartridge 130.

Once the cartridge 130 is inserted within the compartment 216 and the cannula 170 is secured within the connector 220, the injector device 200 is ready for use to inject substances from within the cartridge 130 into a body structure of a patient, such as the anterior chamber of a patient's eye. To inject the substances, a user may insert the needle 174 of the cannula 170 into the body structure and adjacent a target injection site, and then press distally against the plunger ring 250 to drive substances from within the cartridge 130 out of the nozzle 132, through the cannula 170, and out of the distal end of the needle 172.

With reference now to FIGS. 3A and 3B, perspective side views of another injector device 300 are illustrated, according to certain embodiments of the present disclosure. In FIG. 3A, the injector device 300 is shown in an unassembled, or disassembled, state; meanwhile, in FIG. 3B, the injector device 300 is shown in an assembled state. In both FIGS. 3A and 3B, the injector device 300 is shown relative to the cartridge 130 containing a substance for injection and the corresponding cannula 170. For clarity, FIGS. 3A and 3B are described together herein.

As shown in FIGS. 3A and 3B, the injector device 300 is substantially similar in arrangement and function to the injector device 200. Accordingly, like components and features are indicated by like reference numerals to FIGS. 2A and 2B, and are omitted from the description below.

Generally, the injector device 300 is distinguished from the injector device 200 by the absence of the plunger assembly 240. The injector device 300 may therefore be referred to as a “simplified” version of the injector device 200. Instead, the most proximal point of the injector device 300 is the body flange(s) 226, through which the proximal opening 218 is disposed. With the absence of the plunger assembly 240, the proximal opening 218 of the body 310 is configured to accommodate and allow a plunger 346 of the cartridge 130 to pass therethrough. Accordingly, the injector device 300 may be utilized with cartridges 130 in the form of syringes having their own plungers 346. In the illustrated embodiments, the plunger 346 is similar in form and function to the plungers of injector devices 100 and 200. For example, the plunger 346 includes a rod 348 and a plunger flange 350, similar to the rods and plunger flanges described above, to facilitate actuation of the piston 136 of the cartridge 130. However, unlike the previous examples, the rod 348 is directly coupled with the piston 136.

Assembly and use of the injector device 300 is similar to that of the injector device 200. A user (e.g., a surgeon or surgical assistant) first inserts the cartridge 130, nozzle 132 first, through the proximal opening 218 or the window 228 of the body 210 and into the compartment 216. The user then slides the cartridge 130 distally through the compartment 216 such that the nozzle 132 is received in the connector 220. Engagement of one or more surfaces of the cartridge 130 with the connector 220 or another component of the body 210 may produce an audible indication (e.g., a sound) for the user to confirm proper and complete insertion of the cartridge 130 into the compartment 216.

Prior to or after insertion of the cartridge 130, the user secures the cannula 170 to the connector 220 of the body 210. For example, the user may screw the cannula 170 into the connector 220 in examples where the connector includes threads 224. Similar to the above, engagement of one or more surfaces of the cannula 170 with the connector 220 may produce an audible indication for the user to confirm proper fastening of the cannula 170 into the connector 220, and/or proper attachment of the cannula 170 with the cartridge 130.

Once the cartridge 130 is inserted within the compartment 216 and the cannula 170 is secured within the connector 220, the injector device 200 is ready for use to inject substances from within the cartridge 130 into a body structure of a patient, such as the anterior chamber of a patient's eye. To inject the substances, a user may insert the needle 174 of the cannula 170 into the body structure and adjacent a target injection site, and then press distally against the plunger flange 350 to drive substances from within the cartridge 130 out of the nozzle 132, through the cannula 170, and out of the distal end of the needle 172.

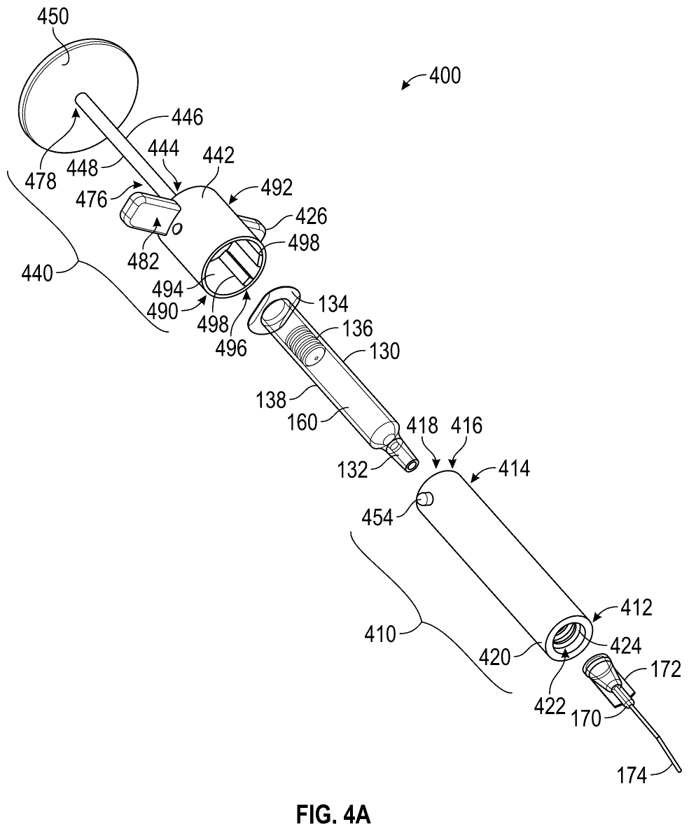

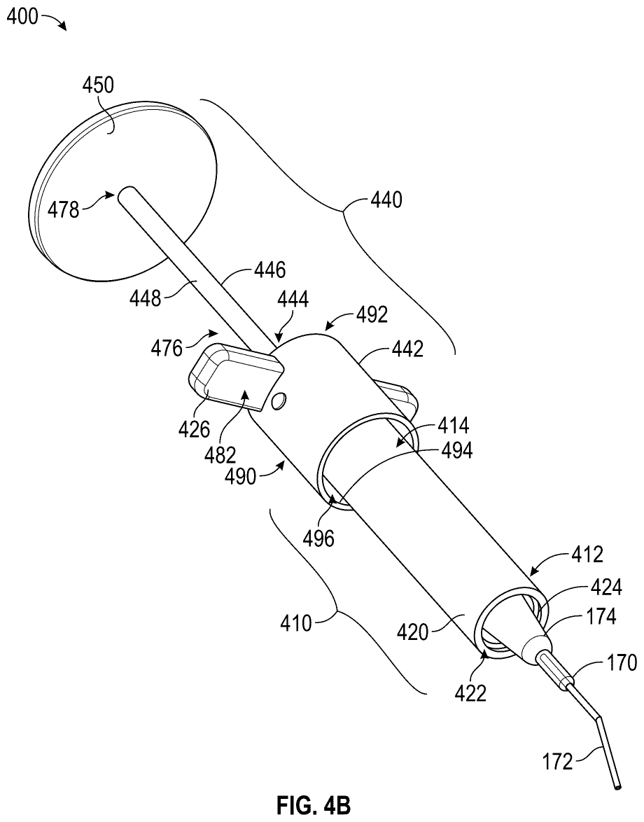

FIG. 4A is a perspective side view of another injector device 400 for injecting substances, according to certain embodiments of the present disclosure. FIG. 4B is a second perspective side view of the injector device 400, according to certain embodiments of the present disclosure. In FIG. 4A, the injector device 400 is shown in an unassembled, or disassembled, state; meanwhile, in FIG. 4B, the injector device 400 is shown in an assembled state. In both FIGS. 4A and 4B, the injector device 400 is shown relative to the cartridge 130 containing a substance for injection and the corresponding cannula 170. For clarity, FIGS. 4A and 4B are described together herein.

As shown in FIGS. 4A and 4B, the injector device 400 includes a body 410 and a plunger assembly 440, similar to the embodiments illustrated in FIGS. 1A-2B. The body 410 is configured to receive at least a distal portion of the cartridge 130 therein, while the plunger assembly 440 is configured to slide over at least a proximal portion of the cartridge 130 and secure the cartridge 130 in the body 410.

In the illustrated embodiments, the body 410 has a generally tubular or cylindrical shape extending between a distal end 412 and a proximal end 414 of the body 410. It should be appreciated that the shape shown relative to the body 410 is merely illustrative. In various embodiments, the body 410 can have any suitable geometry, shape, and/or size including, for example, tapered shapes, ovaloid shapes, and/or the like. Like with other injector devices described herein, the body 410 is formed of any suitable material that facilitates sterilization and re-use of the injector device 400. In certain embodiments, the body 410 is formed of a surgical grade metal or metal alloy, such as stainless steel, titanium, tungsten carbide, carbon steel, and the like. In certain embodiments, the body 410 is formed of a sterilizable thermoplastic or resin-based material.

The body 410 has a compartment 416 formed therein for receiving at least a distal portion of the cartridge 130, including the nozzle 132. The compartment 416 extends from a proximal opening 418 at the proximal end 414 of the body 410 to a distal opening 422 at the distal end 412, or a point near the distal end 412. The compartment 416 is generally shaped and sized to correspond with a shape and size of the cartridge 130. Accordingly, the compartment 416 may have inner dimensions (e.g., an inner diameter, width, or length) substantially similar to outer dimensions (e.g., an outer diameter, width, or length) of the cartridge 130 to facilitate a secure fit of the cartridge 130 within the body 410. In the embodiments of FIGS. 4A and 4B, the cartridge 130 is received by the compartment 416 through the proximal opening 418. Accordingly, the proximal opening 418 may have a cross-section (e.g., cross-sectional size and shape) similar to that of the compartment 416 and/or the cartridge 130.

The body 410 also includes a connector 420 disposed at the distal end 412 of the body 410 that forms the distal opening 422 extending between the compartment 416 and an exterior of the body 410. In certain embodiments, the connector 420 is simply an extension of the body 410. The connector 420 facilitates fluid connection of the cartridge 130 to the cannula 170 when the cartridge 130 is fully received in the compartment 416. Accordingly, the connector 420 is configured to receive at least a portion of a nozzle 132 of the cartridge 130 and at least a portion of a hub 172 of the cannula 170 through the distal opening 422. In certain embodiments, the distal opening 422 has inner dimensions (e.g., an inner diameter or width) that are smaller than those of the compartment 416 to facilitate a secure fit of the nozzle 132 and hub 172. In certain embodiments, the connector 420 includes one or more threads 424 and/or other features disposed along inner surfaces of the connector 420 and surrounding the distal opening 422. In such embodiments, the thread(s) 424 and/or other feature(s) of the connector 420 are configured to mate with corresponding features on the hub 172 to secure the cannula 170 to the body 410 while the cannula 170 is connected to the cartridge 130 received in the compartment 416. In certain embodiments, fully securing the cannula 170 against the connector 320 may produce an audible indication for the user that confirms proper fastening of the components.

In certain embodiments, in addition to the connector 420, the injector device 400 may include a secondary locking mechanism for the cannula 170. For example, the injector device 400 may include a claw or clamp that is disposed around the distal end of the connector 420 and that when compressed, enables the cannula 170 to be inserted into the distal opening 422. When the claw or clamp is released, the claw or clamp “hugs” the cannula 170 to secure the cannula 170 in the distal opening 422 and prevent the cannula 170 from distally separating from the injector device 400.

The body 410 further includes one or more locking features 454 disposed at the proximal end 414 for securing the body 410 to the plunger assembly 440. Such locking features 454 may include snap-fit protrusions (or grooves) that are disposed on an internal or external surface of the body 410 and are configured to engage with corresponding features on an endcap 442 of the plunger assembly 440. In certain embodiments, fully securing the endcap 442 against the body 410 may produce an audible indication for the user that confirms proper fastening of the components. In certain embodiments, the one or more locking features 454 may alternatively or additionally function as alignment features for aligning the body 410 with the plunger assembly 440 during assembly.

In certain embodiments, a window may extend along a length of the body 410 to facilitate viewing by the surgeon or surgical assistant of the cartridge 130 disposed in the injector device 400, the type of substance disposed in the cartridge 130, and/or of an amount of substance remaining in the cartridge 130, when the cartridge 130 is received in the compartment 416. In certain embodiments, as an alternative or in addition to the window, the body 410 may be formed of a transparent or translucent material to facilitate visibility of an inserted cartridge 130.

In certain embodiments, a shape of the body 410 (or another component of the injector device 400) indicates or represents the type of cartridge 130 for use with the injector device 400, and/or a type of substance in the cartridge 130. In certain embodiments, the body 410 (or another component of the injector device 400) includes one or more visual indicators that indicate, or represent, the type of cartridge 130 and/or substance in the cartridge 130. For example, the body 410 can include a marking or other graphic on an external surface thereof to indicate the type of cartridge 130 and/or substance disposed therein. In certain embodiments, one or more components of the injector device 400 are color-coded to indicate the type of cartridge 130 and/or substance disposed therein. For example, the body 410 can include a color-coded ring (e.g., disposed around an external surface thereof) to indicate the type of cartridge 130 and/or substance for use with the injector device 400.

The plunger assembly 440 includes an endcap 442 and a plunger 446 movably disposed through the endcap 442. Similar to the body 410, the endcap 442 and the plunger 446 are formed of any suitable material(s) that facilitate sterilization and re-use of the injector device 400. In certain embodiments, the endcap 442 and/or the plunger 446 are formed of a surgical grade metal or metal alloy, such as stainless steel, titanium, tungsten carbide, carbon steel, and the like. In certain embodiments, the endcap 442 and/or the plunger 446 are formed of a sterilizable thermoplastic or resin-based material. In certain embodiments, the body 410, the endcap 442, and the plunger 446 are formed of the same material. In certain other embodiments, the body 410, the endcap 442, and/or the plunger 446 are formed of a different material from one or both of the other components.

The endcap 142 includes a suitably-shaped capping member configured to slide over and fasten against the proximal end 414 of the body 410 after insertion of the cartridge 130 into the compartment 416. In the embodiments of FIGS. 4A and 4B, the endcap 442 resembles a cylindrical push-pull cap having a distal end 490, a proximal end 492, and a compartment 494. The compartment 494 is configured to receive the proximal end 414 of the body 410 via a distal opening 496 at the distal end 490 during assembly of the injector device 400. Accordingly, the distal opening 496 and compartment 494 are generally shaped and sized to correspond with a shape and size of the proximal end 414 of the body 410. Meanwhile, an aperture 444 is formed in the distal end 490 through which the plunger 446 is slidably disposed. Fastening the endcap 442 against the body 410 secures the cartridge 130 within the body 410, and further positions the plunger 446 to engage with the piston 136 of the cartridge 130.

As noted above, in certain embodiments, the endcap 442 includes one or more locking features 498 disposed on an internal or external surface thereof for securing the endcap 442 to the body 410. Such locking features 498 may correspond with, and engage with, the locking features 454 at the proximal end 414 of the body 410. In the embodiments of FIGS. 4A and 4B, the locking features 498 include snap-fitting grooves arranged on an internal surface of the endcap 442, while the locking features 454 include snap-fitting protrusions arranged on an external surface of the body 410. However, it is further contemplated that the locking features 498 may include protrusions while the locking features 454 include grooves. Other types of features for each of the locking features 498 and 454 are also contemplated. Still further, the one or more locking features 498 may alternatively or additionally function as alignment features for aligning the plunger assembly 440 with the body 410 during assembly. And, in certain embodiments, fully securing the endcap 442 against the body 410 may produce an audible indication for the user that confirms proper fastening of the components.

The endcap 442 further includes one or more endcap flanges 426 disposed at or near the proximal end 492 and extending laterally, or radially, outward from the endcap 442. A distal surface 482 of each endcap flange 426 facilitates grasping of the injector device 400 by the fingers of a surgeon or surgical assistant during a surgical operation, such as a cataract procedure or other ophthalmic procedure. Thus, in certain examples herein, an endcap flange 426 may be referred to as a “flinger flange.” In certain embodiments, a singular endcap flange 426 is disposed around a portion of, or an entirety of, a cross-sectional outer perimeter (e.g., circumference) of the endcap 442; in certain other embodiments, two or more endcap flanges 426 are each disposed around a portion of the cross-sectional outer perimeter of endcap 442.

The plunger 446 includes a rod 448 and a plunger flange 450 fixedly coupled to a proximal end 478 of the rod 448. The rod 448 includes an elongated member configured to slide axially through the aperture 444 of the endcap 442 upon actuation by a user. When the endcap 442 is fastened against the body 410, a distal end 476 of the rod 448 is configured to translate axially through the compartment 416 of the body 410 and engage the piston 136 of the cartridge 130 for dispensing fluids from the cartridge 130. In certain embodiments, the distal end 476 of the rod 448 includes a stopper, or flange, for preventing the plunger 446 from sliding proximally out of the aperture 444, similar to the example in FIG. 1B. In such embodiments, the stopper may directly engage with the piston 134 of the cartridge 130 during use.

The plunger flange 450 provides a surface upon which a user (e.g., a surgeon) may press to actuate the rod 448. Typically, the user will press against the plunger flange 450 with their thumb, and so the plunger flange 450 may be referred to as a “thumb flange” in certain examples herein. In certain examples, the plunger flange 450 may include one or more textured, contoured, and/or curved surfaces for improved gripping thereof by the user. In still further embodiments, the plunger flange 450 may be replaced by another feature to facilitate actuation of the rod 448, such as a ring as shown in the embodiments of FIGS. 2A and 2B.

To assemble and use the injector device 400, a user (e.g., a surgeon or surgical assistant) first inserts the cartridge 130, nozzle 132 first, through the proximal opening 418 of the body 410 and into the compartment 416. The user then slides the cartridge 130 distally through the compartment 416 such that the nozzle 132 is received in the connector 420. Engagement of one or more surfaces of the cartridge 130 with the connector 420 or another component of the body 410 may produce an audible indication (e.g., a sound) for the user to confirm proper and complete insertion of the cartridge 130 into the compartment 416. Once the cartridge 130 is fully inserted into the compartment 416 of the body 410, the user slides the plunger assembly 440 over the proximal end 414 of the body 410, and presses the endcap 442 distally to engage the locking features 454 and 498 and secure the plunger assembly 440 onto the body 410. Again, locking of the endcap 442 against the body 410 (e.g., full engagement of the locking features 454 and 498) may produce an audible indication for the user to confirm proper fastening of the components.

Prior to or after insertion of the cartridge 130 and fastening of the plunger assembly 440 and the body 410, the user secures the cannula 170 to the connector 420 of the body 410. For example, the user may screw the cannula 170 into the connector 420 in examples where the connector includes threads 424. Similar to the above, engagement of one or more surfaces of the cannula 170 with the connector 420 may produce an audible indication for the user to confirm proper fastening of the cannula 170 into the connector 420, and/or proper attachment of the cannula 170 with the cartridge 130.

Once the cartridge 130 is inserted within the compartment 416, the plunger assembly 440 is fastened against the body 410, and the cannula 170 is secured within the connector 420, the injector device 400 is ready for use to inject substances from within the cartridge 130 into a body structure of a patient, such as the anterior chamber of a patient's eye. To inject the substances, a user may insert the needle 174 of the cannula 170 into the body structure and adjacent a target injection site, and then press distally against the plunger flange 450 to drive substances from within the cartridge 130 out of the nozzle 132, through the cannula 170, and out of the distal end of the needle 172.

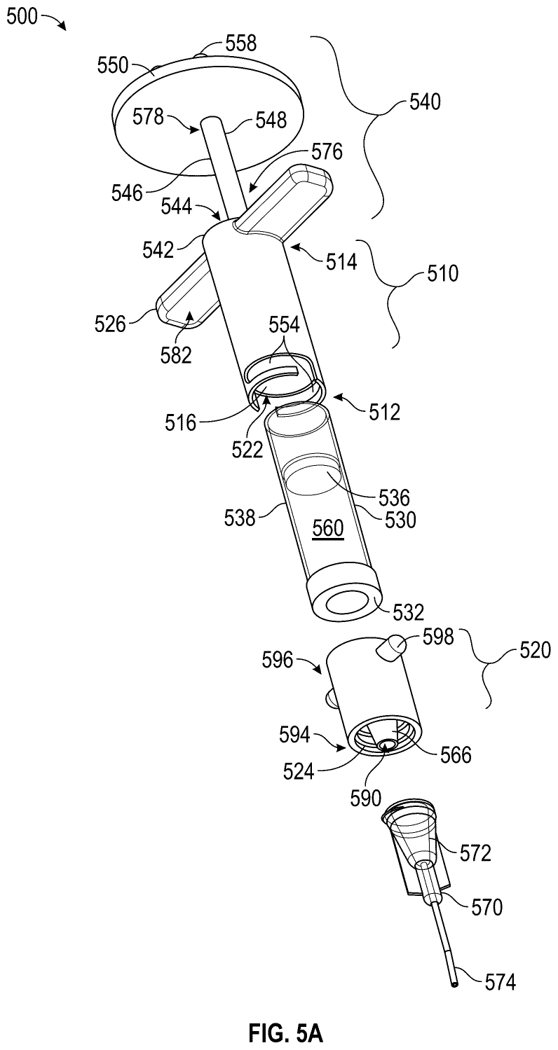

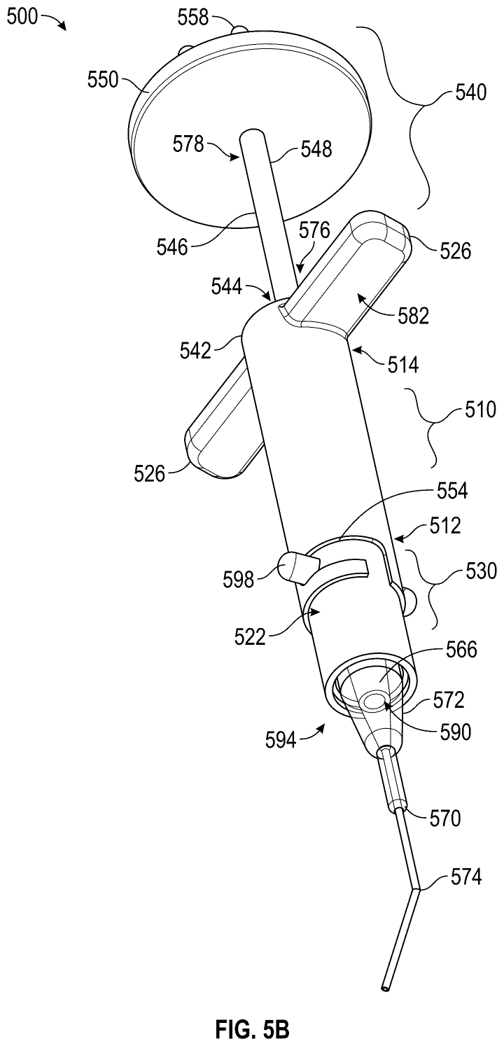

FIG. 5A is a perspective side view of another injector device 500 for injecting substances, according to certain embodiments of the present disclosure. FIG. 5B is a second perspective side view of the injector device 500, according to certain embodiments of the present disclosure. In FIG. 5A, the injector device 500 is shown in an unassembled, or disassembled, state; meanwhile, in FIG. 5B, the injector device 500 is shown in an assembled state. In both FIGS. 5A and 5B, the injector device 500 is shown relative to a cartridge 530 containing a substance for injection and a corresponding cannula 570. For clarity, FIGS. 5A and 5B are described together herein.

As shown in FIGS. 5A and 5B, the injector device 500 includes a body 510, a plunger assembly 540 integrated with the body 510, and a connector 520. The body 510 is configured to receive at least a proximal portion of the cartridge 530 therein, while the connector 520 is configured to slide over at least a distal portion of the cartridge 530 and engage with the body 510 to secure the cartridge 530 between the connector 520 and the body 510.

As noted above, the body 510 is integrated with the plunger assembly 540, and so a proximal end 514 of the body 510 is fixedly coupled to the plunger assembly 540. Meanwhile, a distal end 512 of the body 510 is configured to removably couple to the connector 520. In the illustrated embodiments, the body 510 has a generally tubular or cylindrical shape extending between the distal end 512 and the proximal end 514. It should be appreciated that the shape shown relative to the body 510 is merely illustrative. In various embodiments, the body 510 can have any suitable geometry, shape, and/or size including, for example, tapered shapes, ovaloid shapes, and/or the like.

The body 510 has a compartment 516 formed therein for receiving at least a proximal portion of the cartridge 530. The compartment 516 extends from a distal opening 522 at the distal end 512 of the body 510 to the plunger assembly 540 at the proximal end 514, or a point near the proximal end 514. The compartment 516 is generally shaped and sized to correspond with a shape and size of the cartridge 530. Accordingly, the compartment 516 may have inner dimensions (e.g., an inner diameter, width, or length) substantially similar to outer dimensions (e.g., an outer diameter, width, or length) of the cartridge 530 to facilitate a secure fit of the cartridge 530 within the body 510. In the embodiments of FIGS. 5A and 5B, the cartridge 530 is received by the compartment 516 through the distal opening 522. Accordingly, the distal opening 522 may have a cross-section (e.g., cross-sectional size and shape) similar to that of the compartment 516 and/or the cartridge 530.

The body 510 further includes one or more locking features 454 at the distal end 512 for securing the body 510 to the connector 520. Generally, the locking features 554 are configured to engage and mate with corresponding features on the connector 520. In the embodiments of FIGS. 5A and 5B, the locking features 545 include two L-shaped cutouts that are configured to receive and secure corresponding protrusions 598 disposed on the connector 520 during assembly. However, other types of locking features 545 are also contemplated, including snap-fit protrusions or grooves, and the like. In certain embodiments, fully securing the connector 520 against the body 410 may produce an audible indication for the user that confirms proper fastening of the components.

In certain embodiments, a window may extend along a length of the body 510 to facilitate viewing by the surgeon or surgical assistant of the cartridge 530 disposed in the injector device 500, the type of substance disposed in the cartridge 530, and/or an amount of substance remaining in the cartridge 530, when the cartridge 530 is received in the compartment 516. In certain embodiments, as an alternative or in addition to the window, the body 510 (and/or other components of the injector device 500) may be formed of a transparent or translucent material to facilitate visibility of an inserted cartridge 530.

In certain embodiments, a shape of the body 510 (or another component of the injector device 500) indicates or represents the type of cartridge 530 for use with the injector device 500, and/or a type of substance in the cartridge 530. In certain embodiments, the body 510 (or another component of the injector device 500) includes one or more visual indicators that indicate, or represent, the type of cartridge 530 and/or substance in the cartridge 530. For example, the body 510 can include a marking or other graphic on an external surface thereof to indicate the type of cartridge 530 and/or substance disposed therein. In certain embodiments, one or more components of the injector device 500 are color-coded to indicate the type of cartridge 530 and/or substance disposed therein. For example, the body 510 can include a color-coded ring (e.g., disposed around an external surface thereof) to indicate the type of cartridge 530 and/or substance for use with the injector device 500.

The plunger assembly 540 includes an endcap 542 and a plunger 546 movably disposed through the endcap 542. The endcap 542 caps, or covers, the proximal end 514 of the body 510. In the embodiments of FIGS. 5A and 5B, the endcap 542 is fixedly attached to the body 510. An aperture 544 is formed in the endcap 542 through which the plunger 546 is slidably disposed.

The plunger 546 includes a rod 548 and a plunger flange 550 fixedly coupled to a proximal end 578 of the rod 548. The rod 548 includes an elongated member configured to slide axially through the aperture 544 of the endcap 542 upon actuation by a user. Accordingly, a distal end 576 of the rod 548 is configured to translate axially through the compartment 516 of the body 510 and engage a piston 536 of the cartridge 530 when the injector device 500 is assembled. In certain embodiments, the distal end 576 of the 5od 448 includes a stopper, or flange, for preventing the plunger 546 from sliding proximally out of the aperture 544, similar to the example in FIG. 1B. In such embodiments, the stopper may directly engage with the piston 536 of the cartridge 530 to dispense substances from the cartridge 530 during use.

The plunger flange 550 provides a surface upon which a user (e.g., a surgeon) may press to actuate the rod 548. Typically, the user will press against the plunger flange 550 with their thumb, and so the plunger flange 550 may be referred to as a “thumb flange” in certain examples herein. In certain examples, the plunger flange 550 may include one or more textured, contoured, and/or curved surfaces 558 for improved gripping thereof by the user. In still further embodiments, the plunger flange 550 may be replaced by another feature to facilitate actuation of the rod 548, such as a ring as shown in the embodiments of FIGS. 2A and 2B.

The endcap 542, or in certain embodiments, the proximal end 514 of the body 510, further includes one or more finger flanges 526 extending laterally, or radially, outward from the endcap 542 or body 510. A distal surface 582 of each finger flange 526 facilitates grasping of the injector device 500 by the fingers of a surgeon or surgical assistant during a surgical operation, such as a cataract procedure or other ophthalmic procedure. In certain embodiments, a singular finger flange 526 is disposed around a portion of, or an entirety of, a cross-sectional outer perimeter (e.g., circumference) of the endcap 542 or body 510; in certain other embodiments, two or more finger flanges 526 are each disposed around a portion of the cross-sectional outer perimeter of endcap 542 or body 510.

The connector 520 removably couples to the distal end 512 of the body 510. When the injector device 500 is assembled, the connector 520 facilitates fluid connection of the cartridge 530 disposed between the body 510 and the connector 520 to the cannula 570. To facilitate the connection of the cartridge 530 to the cannula 570, the connector 520 has an opening 590 (e.g., an aperture) extending between a distal end 592 and a proximal end 594 of the connector 520. In certain embodiments, the connector 520 includes an adapter 566 centrally disposed within the connector 520 and through which the opening 590 extends. The adapter 566 may resemble and function as a nozzle for the cartridge 530, which may not have a nozzle in certain embodiments, as shown in FIGS. 5A and 5B. In such embodiments, the adapter 566 acts an intermediary between the cartridge 530 and the cannula 570. Accordingly, the adapter 566 may be configured to fluidly couple with both the cartridge 530 and a hub 572 of the cannula 570. In certain other embodiments, however, the opening 590 is configured to receive at least a portion of a nozzle of a cartridge, which can be fluidly connected with the hub 572 of the cannula 570 in the opening 590.

In certain embodiments, the connector 520 includes one or more threads 524 and/or other features disposed along inner surfaces of the connector 520 and surrounding the opening 590 and/or adapter 566. In such embodiments, the thread(s) 524 and/or other feature(s) of the connector 520 are configured to mate with corresponding features on the hub 572 to secure the cannula 570 to the injector device 500 during use. In certain embodiments, fully securing the cannula 570 against the connector 520 may produce an audible indication for the user that confirms proper fastening of the components.

In certain embodiments, in addition to the connector 520, the injector device 500 may include a secondary locking mechanism for the cannula 570. For example, the injector device 500 may include a claw or clamp that is disposed around the distal end of the connector 520 and that when compressed, enables the cannula 570 to be inserted into the distal opening 522. When the claw or clamp is released, the claw or clamp “hugs” the cannula 570 to secure the cannula 570 in the distal opening 522 and prevent the cannula 570 from distally separating from the injector device 500.

As noted above, in certain embodiments, the connector 520 includes one or more locking features 598 disposed on an internal or external surface thereof for securing the connector 520 to the body 510. Such locking features 598 may correspond with, and engage with, the locking features 554 at the distal end 512 of the body 510. In the embodiments of FIGS. 5A and 5B, the locking features 598 include protrusions arranged on an external surface of the connector 520 at a proximal end 596, while the locking features 554 include L-shaped cutouts formed through a sidewall of the body 510. In such embodiments, the protrusions on the connector 520 may be translated through the both arms of the L-shaped cutouts to align and secure the connector 520 with the body 510, as further described below. However, other types of features for each of the locking features 598 and 554 are also contemplated. In certain embodiments, fully securing the connector 520 against the body 510 may produce an audible indication for the user that confirms proper fastening of the components.

Generally, the body 510, the endcap 542, the plunger 546, and the connector 520 are formed of any suitable material(s) that facilitate sterilization and re-use of the injector device 500. In certain embodiments, one or more of the body 510, the endcap 542, the plunger 546, and the connector 520 are formed of a surgical grade metal or metal alloy, such as stainless steel, titanium, tungsten carbide, carbon steel, and the like. In certain embodiments, one or more of the body 510, the endcap 542, the plunger 546, and the connector 520 are formed of a sterilizable thermoplastic or resin-based material. In certain embodiments, the body 510, the endcap 542, the plunger 546, and the connector 520 are formed of the same material. In certain other embodiments, the body 510, the endcap 542, the plunger 546, and/or the connector 520 are formed of a different material from one or more of the other components.

The cartridge 530 can include any suitable type of cartridge or receptacle for storing and delivering substances for injection. In certain embodiments, such as shown in FIGS. 5A and 5B, the cartridge 530 resembles a sealed cylindrical container. However, other types of cartridges are also contemplated for use with the injector device 500, including cartridges similar to the cartridge 130. Generally, the cartridge 530 includes a body 538, a sealing member 532 disposed at a distal end of the body 538 (e.g., instead of a nozzle), and the piston 536 disposed within an internal compartment 560 of the cartridge 530. The piston 536 engages with the rod 548 and/or the stopper of the plunger 546 to facilitate dispensing of substances from the cartridge 530, and thus, the injector device 500. The piston 536 may also function along with the sealing member 532 to create an airtight seal around the substance(s) disposed in the internal compartment during storage of the cartridge 530. Meanwhile, the distal end of the cartridge 530, including the sealing member 532, is configured to fluidly couple with the hub 572 of the cannula 570 through the connector 520 during use. In certain embodiments, the sealing member 532 may be broken or punctured to allow substances to pass from the internal compartment 560 to the cannula 570. In such embodiments, the adapter 566 may have one or more structures configured to puncture the sealing member 532 upon securing the connector 520 to the body 510 when the cartridge 520 is disposed in the compartment 516.

In certain embodiments, a type or shape of the cartridge 530 indicates or represents the type of substance contained within the cartridge 530. In certain embodiments, the cartridge 530 includes one or more visual indicators that indicate, or represent, the type of substance contained within the cartridge 530. For example, the cartridge 530 can include a marking or other graphic on an external surface thereof to indicate the type of substance contained therein. In certain embodiments, one or more components of the cartridge 530 are color-coded to indicate the type of substance contained within the cartridge 530. For example, the cartridge 530 can include a color-coded ring around an external surface thereof to indicate the type of substance contained therein. Generally, visual indicators and/or color-coded components on the cartridge 530 may be visible through the window of the body 510 to facilitate viewing by the surgeon or surgical assistant when the cartridge 530 is disposed in the injector device 500.

The cannula 570 includes the hub5 and a needle 574 through which substances from the cartridge 530 are flowed during injection. Generally, the cannula 570 may include any suitable cannula type and size, such as a cannula having a 20-gauge, 21-gauge, 22-gauge, 23-gauge, 24-gauge, 25-gauge, 26-gauge, 27-gauge, 28-gauge, 29-gauge, 30-gauge, 31-gauge, 32-guage, 33-gauge, or smaller or larger diameter needle 574.

To assemble and use the injector device 500, a user (e.g., a surgeon or surgical assistant) first inserts the cartridge 530, proximal end first, through the distal opening 522 of the body 510 and into the compartment 516. Engagement of one or more surfaces of the cartridge 530 with internal surfaces of the body 510 or the plunger assembly 540 may produce an audible indication (e.g., a sound) for the user to confirm proper and complete insertion of the cartridge 530 into the compartment 516.

Once the cartridge 530 is fully inserted into the compartment 516, the user secures the connector 520 to the distal end 512 of the body 510. In the embodiments of FIGS. 5A and 5B, to fasten the connector 520 to the body 510, the user first aligns and proximally slides the protrusions 598 of the connector 520 into a first arm of the L-shaped cutouts 554 of the body 510. Once the protrusions 598 reach a most proximal position along the L-shaped cutouts 554, the user rotates the connector 520 and/or the body 510 relative to each other to slide the protrusion 598 along a second arm of the L-shaped cutouts 554 and lock the connector 520 against the body 510. In certain embodiments, locking of the connector 520 against the body 510 may produce an audible indication for the user to confirm proper fastening of the components.

Prior to or after insertion of the cartridge 530 and fastening of the connector 520 and the body 510, the user secures the cannula 570 to the connector 520. For example, the user may screw the cannula 570 into the connector 520 in examples where the connector 520 includes threads 524. Similar to the above, engagement of one or more surfaces of the cannula 570 with the connector 520 may produce an audible indication for the user to confirm proper fastening of the cannula 570 into the connector 520.

Once the cartridge 530 is inserted within the compartment 516, the connector 520 is fastened against the body 510, and the cannula 570 is secured within the connector 520, the injector device 500 is ready for use to inject substances from within the cartridge 530 into a body structure of a patient, such as the anterior chamber of a patient's eye. To inject the substances, a user may insert the needle 574 of the cannula 570 into the body structure and adjacent a target injection site, and then press distally against the plunger flange 550 to drive substances from within the cartridge 530 through the cannula 570 and out of the distal end of the needle 572.

In summary, the present disclosure provides sterilizable and re-usable devices for injecting substances (including, without limitation, viscoelastics, liquids, gels, solutions, emulsions, suspensions, other fluids, and the like) during surgical procedures. In certain embodiments, the devices disclosed herein can be utilized for injection or application of ophthalmic substances into and/or onto a patient's eye. Examples of ophthalmic substances include, without limitation, ophthalmic viscoelastic devices (OVDs), ophthalmic medicaments such as vascular endothelial growth factor (VEGF) inhibitors, antibiotics, anesthetics, and steroids, other ophthalmic gels and solutions, and the like. However, the utilization of such devices with other types of treatment substances and/or for injection or application of other body structures is also contemplated.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

What is claimed is:

1. A re-usable injection device, comprising:
a body, comprising:
a compartment disposed within the body and configured to receive a cartridge containing one or more substances;
a connector disposed at a distal end of the body and configured to simultaneously receive a hub of a cannula and a nozzle of the cartridge to facilitate connection of the cannula and the cartridge when the cartridge is disposed in the compartment, the connector further comprising a feature for mating with the hub to secure the cannula to the connector when the cannula is connected to the cartridge; and
a finger flange disposed at a proximal end of the body, the finger flange configured to contact a flange of the cartridge when the cartridge is disposed in the compartment; and
a plunger assembly coupled to the proximal end of the body, the plunger assembly comprising:
a cap configured to secure the cartridge when the cartridge is disposed in the compartment; and
a plunger movably disposed through the cap and configured to engage with a piston of the cartridge to dispense fluids from the cartridge when actuated by a user.

2. The injection device of claim 1, wherein the body and the plunger assembly are formed of a sterilizable material to facilitate re-use of the injection device.

3. The injection device of claim 2, wherein the body and the plunger assembly are formed of a metal material comprising at least one of stainless steel, titanium, tungsten carbide, or carbon steel.

4. The injection device of claim 2, wherein the body and the plunger assembly are formed of a thermoplastic material or a resin-based material.

5. The injection device of claim 1, wherein the plunger assembly is hingedly coupled to the proximal end of the body to facilitate insertion and removal of the cartridge into and out of the body via a proximal opening of the body.

6. The injection device of claim 1, wherein the plunger assembly is fixedly coupled to the proximal end of the body via a bridge.

7. The injection device of claim 6, wherein the finger flange comprises a slot configured to receive and secure the flange of the cartridge when the cartridge is disposed in the compartment.

8. The injection device of claim 1, wherein the cap comprises a locking feature configured to engage with the body to secure the plunger assembly to the body and to secure the cartridge when the cartridge is disposed in the compartment.

9. The injection device of claim 8, wherein securing the plunger assembly to the body produces an audible indication for the user to confirm the securing of the plunger assembly to the body.

10. The injection device of claim 1, wherein fully inserting the cartridge into the compartment of the body produces an audible indication for the user to confirm the full insertion of the cartridge into the compartment of the body.

11. The injection device of claim 1, wherein securing the cannula to the connector produces an audible indication for the user to confirm securing of the cannula to the connector.

12. The injection device of claim 1, wherein the body comprises a window extending along a length of the body, the window configured to facilitate visual inspection of the cartridge, a type of the one or more substances within the cartridge, or an amount of the one or more substances within the cartridge when the cartridge is disposed in the compartment.

13. A re-usable injection device, comprising:
a body, comprising:
a compartment disposed within the body and configured to receive a cartridge containing one or more substances;
a connector disposed at a distal end of the body and configured to simultaneously receive a hub of a cannula and a nozzle of the cartridge to facilitate connection of the cannula and the cartridge when the cartridge is disposed in the compartment;
a finger flange disposed at a proximal end of the body, the finger flange comprising a slot configured to receive and secure a flange of the cartridge when the cartridge is disposed in the compartment;
an opening disposed at the proximal end of the body and configured to accommodate a plunger of the cartridge; and
a window extending along a length of the body, the window configured to facilitate insertion of the cartridge into the compartment and visual inspection of the cartridge when the cartridge is disposed in the compartment.

14. The injection device of claim 13, wherein the connector further comprises a feature for mating with the hub of the cannula to secure the cannula to the connector when the cannula is connected to the cartridge.

15. The injection device of claim 13, wherein securing the cannula to the connector produces an audible indication for a user to confirm securing of the cannula to the connector.

16. The injection device of claim 13, wherein the body is formed of a sterilizable material to facilitate re-use of the injection device.

17. The injection device of claim 16, wherein the body is formed of a metal material comprising at least one of stainless steel, titanium, tungsten carbide, or carbon steel.

18. The injection device of claim 16, wherein the body is formed of a thermoplastic material or a resin-based material.

19. The injection device of claim 13, wherein fully inserting the cartridge into the compartment of the body produces an audible indication for a user to confirm the full insertion of the cartridge into the compartment of the body.

20. A re-usable injection device, comprising:
a body, comprising:
a compartment disposed within the body and configured to receive a cartridge containing one or more substances; and
a finger flange disposed at a proximal end of the body, the finger flange configured to contact a flange of the cartridge when the cartridge is disposed in the compartment;
a plunger assembly coupled to the proximal end of the body, the plunger assembly comprising:
a cap fixedly coupled to the body; and
a plunger movably disposed through the cap and configured to engage with a piston of the cartridge to dispense fluids from the cartridge when actuated by a user; and
a connector configured to removably couple to a distal end of the body, the cap configured to facilitate connection of a cannula with the cartridge when the cartridge is disposed in the compartment, the connector further comprising a feature for mating with the cannula to secure the cannula to the connector when the cannula is connected to the cartridge.
